(19) United States
(12) Patent Application Publication
(10) Pub. No.: US 2025/0312796 A1
(43) Pub. Date: Oct. 9, 2025

(54) TESTER STAND

(71) Applicant: INTIN INC., Daegu-si (KR)

(72) Inventor: Ji Hoon KIM, Seoul (KR)

(73) Assignee: INTIN INC., Daegu-si (KR)

(21) Appl. No.: 18/962,370

(22) Filed: Nov. 27, 2024

(30) Foreign Application Priority Data
Apr. 5, 2024 (KR) 10-2024-0046796

(51) Int. Cl. B01L 9/00; B01L 3/00

(52) U.S. Cl. CPC B01L 9/00; B01L 3/56

(57) ABSTRACT

Disclosed is a tester stand including: a body having a predetermined height, and a chamber task area and a device task area each formed in a groove shape engraved in an upper surface of the body; a chamber seating portion formed by protruding from a bottom surface of the chamber task area and in which the chamber is placed; a device seating portion having a device insertion groove formed in a bottom surface of the device task area so that a part of the tester is seated therein, and having a downward fastening insertion groove on the bottom surface of the device insertion groove; and a height adjustment portion fitted into the fastening insertion groove so that a support plate is positioned on the bottom surface of the device insertion groove.

### CROSS-REFERENCE TO RELATED APPLICATIONS

This application claims priority to and the benefit of Korean Patent Application No. 10-2024-0046796, filed on Apr. 5, 2024, the disclosure of which is incorporated herein by reference in its entirety.

### FIELD

The present disclosure relates to a stand, and more particularly, to a tester stand that holds both a tester and a chamber, allowing a user to easily inject a liquid substance sample into the chamber and insert the chamber into the tester.

### BACKGROUND

First, the term “liquid substance” hereinafter refers to all types of bodily fluids contained in the human body, such as saliva, blood, semen, sweat, urine, and tears. More broadly, it is defined to include various types of similar liquid substances, including organic and inorganic compounds used in scientific research fields such as medicine, biotechnology, water quality, and the environment.

In general, liquid substance testing devices (hereinafter referred to as “testers”) have been developed and are being provided to test liquid substances. Among these conventional testers, there are testers that receive a liquid substance sample using a chamber that holds the sample, as described in Korean Patent Nos. 10-2288223 and 10-2333043. Here, the term “chamber” collectively refers to all types of devices that hold a liquid substance sample and are inserted into a tester to supply the sample.

A common method of utilizing a chamber in such a tester is for the user to place the chamber on a table or desk, and then supply a liquid substance sample to the chamber using a liquid substance sample supply means from which a liquid substance sample is collected. At this point, since the chamber's thickness is about 1 mm, the user holds the chamber by hand to supply the liquid substance sample.

The user then holds the tester after placing the tester back on a desk or table, or holds the tester in mid-air, and then inserts the chamber containing a fluid sample into the tester.

However, because the user holds the chamber or tester by hand during this process to perform a task, they may feel uncomfortable, and accidents such as dropping the chamber or tester may occur, leading to damage.

In addition, the tester using the chamber allows the user to observe a liquid substance sample contained in the tester through a camera lens of a mobile phone when the liquid substance sample is contained. However, it is inconvenient to observe the liquid substance sample by pressing the mobile phone against the tester in mid-air or on a table.

### SUMMARY

In view of the above, the present disclosure provides a tester stand that allows a user to easily inject a liquid substance sample into a chamber and to easily insert the chamber into the tester.

The present disclosure also provides a tester stand a tester stand that allows a user to observe a liquid substance sample within a tester through a lens of a mobile phone without holding the mobile phone or the tester.

In one general aspect, there is provided a tester stand including: a body having a predetermined height, and a chamber task area and a device task area each formed in a groove shape engraved in an upper surface of the body; a chamber seating portion formed by protruding from a bottom surface of the chamber task area and in which the chamber is placed; a device seating portion having a device insertion groove formed in a bottom surface of the device task area so that a part of the tester is seated therein, and having a downward fastening insertion groove on the bottom surface of the device insertion groove; and a height adjustment portion fitted into the fastening insertion groove so that a support plate is positioned on the bottom surface of the device insertion groove, wherein when an external force is applied, the support plate descends to press against the bottom surface, and when the external force is removed, the support plate rises to return to an initial position thereof.

A non-slip pad portion may be formed in the upper surface of the body to prevent the mobile phone from slipping.

The height adjustment portion may include: a support plate positioned on the bottom surface of the device insertion groove and in close contact with the inserted tester to support the tester; a fastening rod protruding downward from a lower surface of the support plate and inserted into the fastening insertion groove; a plurality of rise-restricting legs protruding downward from the lower surface of the support plate, penetrating through the bottom surface of the device seating portion, and having catches formed at ends of the plurality of rise-restricting legs to engage with a bottom of the device seating portion when the support plate is raised; and an elastic body fitted onto the fastening rod and inserted into the fastening insertion groove 132, wherein the elastic body contracts to generate elasticity when external force is applied, whereas the elastic body raises the support plate with the elasticity when the external force is removed.

The height adjustment portion may further include a fixing plate that protrudes vertically from an edge of the support plate and contacts a side surface of the tester positioned on the support plate to fix the tester.

The chamber seating portion may include: a chamber seating area formed by protruding from the bottom surface of the chamber task area and having a shape identical to an outer shape of the chamber; and a fixing frame formed by protruding along an edge of the chamber seating area to a predetermined height.

The chamber seating portion may further include a fitting groove formed in a shape of a groove in the chamber seating area and into which a protrusion formed in the rear surface of the chamber is fitted.

The chamber seating area may be coated with a first color different from a color of the chamber task area, or a tape or film having the first color is attached to the chamber seating area.

### BRIEF DESCRIPTION OF THE DRAWINGS

FIG. 1 is a top perspective view of a tester stand according to an embodiment of the present disclosure.

FIG. 2 is a rear perspective view of a tester stand according to an embodiment of the present disclosure.

FIG. 3 is a perspective view showing a height difference between a chamber area and a tester area in a tester stand according to an embodiment of the present disclosure.

FIG. 4 is an exploded perspective view of a height adjustment portion in a tester stand according to an embodiment of the present disclosure.

FIG. 5 is a perspective view showing a height adjustment portion mounted on a tester stand according to an embodiment of the present disclosure.

FIG. 6 is a perspective view showing a state in which a cup is mounted on the back of a tester stand according to an embodiment of the present disclosure.

FIG. 7 is a perspective view showing a chamber and a tester mounted in a chamber area and a tester area of a tester stand according to an embodiment of the present disclosure.

FIG. 8 is a perspective view showing a state in which a chamber is inserted into a liquid substance tester in a tester stand according to an embodiment of the present disclosure.

FIG. 9 is a perspective view showing a state in which a mobile phone with a flat lens is placed on a tester stand according to an embodiment of the present disclosure.

FIG. 10 is a perspective view showing a state in which a mobile phone with a protruding lens is placed on a tester stand according to an embodiment of the present disclosure.

### DETAILED DESCRIPTION

Description will now be given in detail according to exemplary embodiments disclosed herein, with reference to the accompanying drawings. For the sake of brief description with reference to the drawings, the same or equivalent components may be provided with the same or similar reference numbers, and description thereof will not be repeated. In addition, in the following description of the embodiments, a detailed description of known functions and configurations incorporated herein will be omitted when it may impede the understanding of the embodiments.

While terms including ordinal numbers, such as “first” and “second,” etc., may be used to describe various components, such components are not limited by the above terms. The above terms are used only to distinguish one component from another.

The singular forms are intended to include the plural forms, unless the context clearly indicates a different meaning.

In this application, the described steps may be carried out in any sequence, except in cases where a clearly defined cause-and-effect relationship necessitates a specific order.

It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

Hereinafter, the present disclosure will be described with reference to the attached drawings.

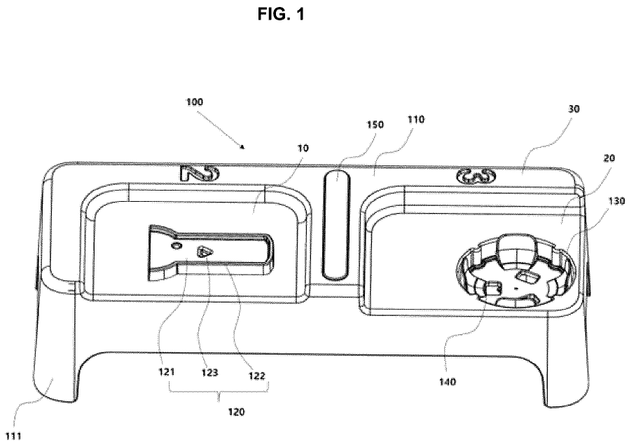

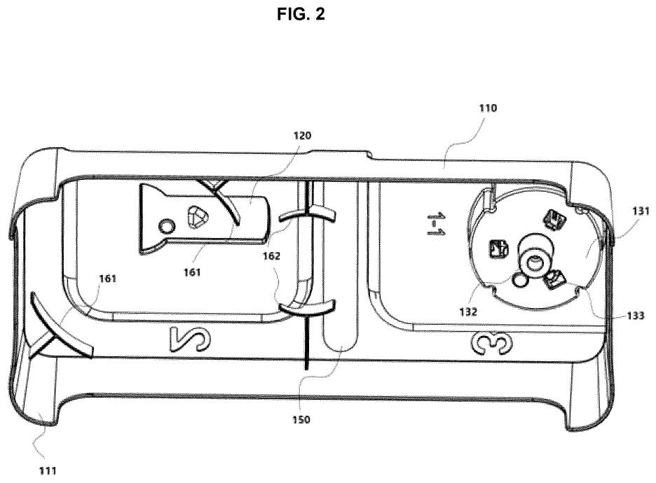

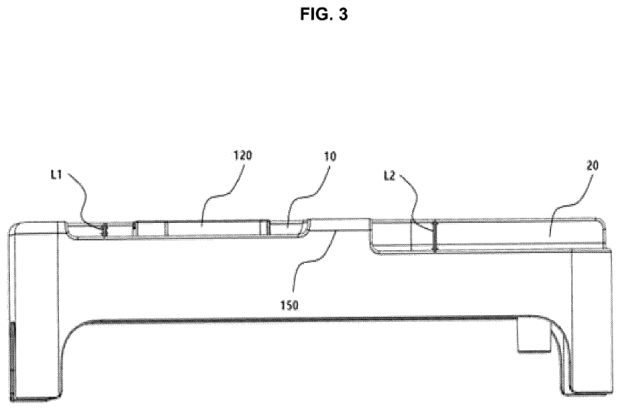

FIG. 1 is a top perspective view of a tester holder according to an embodiment of the present disclosure, FIG. 2 is a rear perspective view of a tester holder according to an embodiment of the present disclosure, and FIG. 3 is a perspective view showing a height difference between a chamber area and a tester area in a tester holder according to an embodiment of the present disclosure.

Referring to FIGS. 1 to 3, a tester stand 100 according to an embodiment of the present disclosure may include a body 110, a chamber seating portion 120, a device seating portion 130, and a height adjustment portion 140.

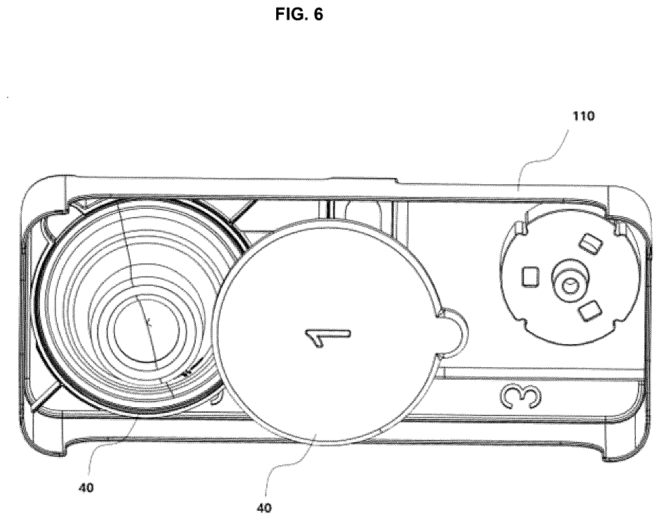

The body 110 is manufactured to be h when placed on the ground or a flat object (i.e., a desk, table, etc.), and to have a predetermined thickness (i.e., height) for a space in which a tester is to be placed and moved vertically. The predetermined thickness may be manufactured to a length that prevents a part of the height adjustment portion 140 from touching the ground or an object on which the stand 100 is placed when the height adjustment portion 140 is lowered vertically. Alternatively, a predetermined thickness may be manufactured to a length that prevents a cup 40 (see FIG. 6) mounted on the rear from touching the ground or an object when the cup 40 mounted on the back is mounted on the rear.

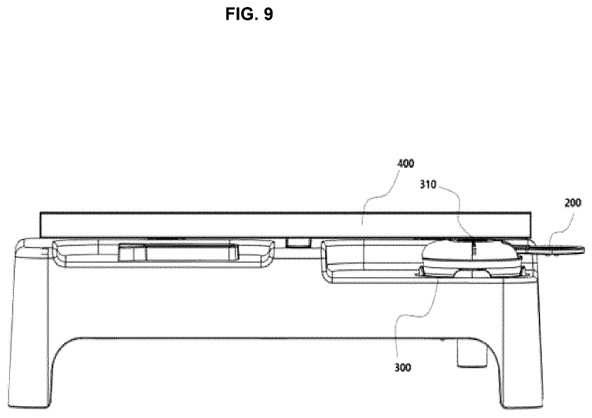

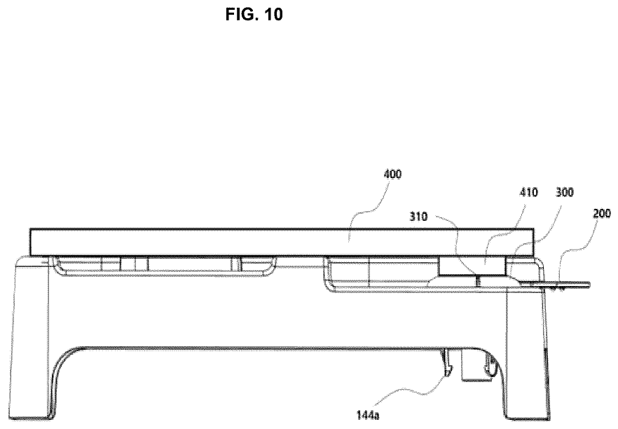

The overall shape of the body 110 may be in the form of a cuboid, considering that a mobile phone is placed on an upper surface of the body 110, but aspects of the present disclosure are not limited thereto. To position a mobile phone 400 (see FIGS. 9 and 10) parallel to the tester placed on the upper surface of the body 110, a mobile phone seating area 30 that comes into contact with the mobile phone is formed in the upper surface of the body 110. The mobile phone seating area 30 is formed flat.

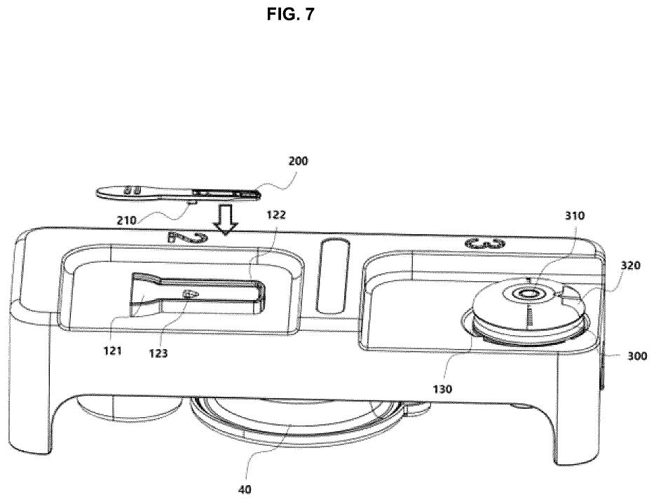

On the upper surface of the body 110, a chamber task area 10 may be formed for a task of placing a chamber 200 (see FIG. 7) by a user and injecting a liquid material sample into the chamber, and a device task area 20 may be formed for a task of placing a tester 300 (see FIG. 7) and inserting the chamber 200 into the tester 300.

The chamber task area 10 is formed as a negative on the upper surface of the body 110 and includes an area where a chamber seating portion 120 is formed. The chamber seating portion 120 is manufactured to have a larger area than the chamber seating portion 120 to facilitate a user's task of placing the chamber 200 by hand or holding the chamber 200 and injecting a liquid material sample into the chamber 200.

The chamber task area 10 may be formed in the shape of a groove so as to be located lower than the mobile phone seating area 30. At this point, the depth of the groove may L1. Furthermore, the chamber task area 10 may have at least one inner face open to the outside to allow the user's hand to freely move to the chamber seating portion 120. At this point, the inner face of the open chamber task area 10 may include one of the two longitudinal side surfaces of the body 110.

The device task area 20 includes an area engraved on the upper surface of the body 110, the area where the device seating portion 130 is formed. The area is manufactured to be larger than the device seating portion 130 to facilitate the user's task of mounting the tester 300 by hand or holding the tester 300 and inserting the chamber 200.

The device task area 20 may be formed in the shape of a groove so as to be located lower than the mobile phone seating area 30. At this point, the depth of the groove may be L1, considering that the thickness of the tester 300 is greater than that of the chamber 200. Of course, in some cases, L1 may be the same length as L2 or longer than L2.

Furthermore, the device task area 20 may have at least one inner face open to the outside so that the user's hand can freely move to the chamber seating portion 120 and insert the chamber 200 into an insertion hole 320 (see FIG. 7) of the tester 300. At this point, the inner face of the open device task area 20 may include one of the two longitudinal side surfaces of the body 110 and may further include either one longitudinal side surface closer to the device task area 20 or the other longitudinal side surface of the body 110.

On the upper surface of the body 110, the number ‘2’ may be formed inside or near the chamber task area 10, and the number ‘3’ may be formed inside or near the device task area 20. The numbers ‘2’ and ‘3’ represent the task sequence, and the number ‘1’ may be formed in the cup 40 as shown in FIG. 6.

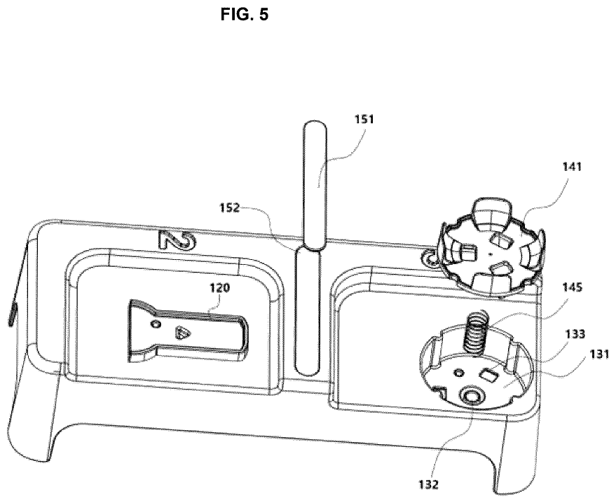

Meanwhile, a non-slip pad portion 150 may be formed in the body 110 to prevent the mobile phone 400 from slipping when placed on the tester stand 100. Referring to FIG. 5, the non-slip pad portion 150 may include a non-slip pad 151 and a pad groove 152 to which the non-slip pad 151 is mounted or attached. Meanwhile, the body 110 may be formed with a leg-shaped support member 111 protruding downward from each corner of a rear surface.

The chamber seating portion 120 is formed within the chamber task area 10, and the chamber 200 is mounted therein. The chamber seating portion 120 may include a chamber seating area 121, a fixing frame 122, and a fitting groove 123.

The chamber seating area 121 may be manufactured in the same shape as an outer shape of the chamber 200, but may be manufactured in at least the same shape as an outer shape of a part of the chamber 200 where a liquid substance sample is contained. In each drawing of the present disclosure, an example is shown in which the chamber seating area 121 is manufactured in the shape of the outer surface of the remaining portion of the chamber 200, except for a part of a grip portion.

The chamber seating area 121 may be formed by protruding from a bottom surface of the chamber task area 10. At this point, a maximum protruding position may be the same as or lower than a position of the mobile phone seating area 30. The entire chamber seating area 121 may be coated with a first color other than white, or a tape or film having the first color may be attached. The reason why the chamber seating area 121 is colored in the first color is that when the body 110 is white, the chamber 200 may not be easily identified because the chamber 200 is transparent.

The fixing frame 122 is formed to protrude along the edge of the chamber seating area 121 to a predetermined height in order to fix the chamber 200 placed in the chamber seating area 121. At this point, the fixing frame 122 may be formed not on at least one of the two longitudinal sides of the chamber seating area 121 so that the chamber 200 can be easily placed in the chamber seating area 121. Of course, the fixing frame 122 may protrude intermittently along the edge of the chamber mounting area 121 to form a space between adjacent protruding portions.

The fitting groove 123 is a groove formed corresponding to a protrusion 210 on the rear surface of the chamber 200. The fitting groove 123 allows the user to confirm that the chamber 200 is completely seated in the chamber seating area 121 by fitting the protrusion 210, and the fitting groove 123 fixes the chamber 200 to the chamber seating area 121. The fitting groove 123 may be omitted in some cases.

The device seating portion 130 is formed within the device task area 20 and is manufactured so that the tester 300 and the height adjustment portion 140 are seated therein. At this point, the device seating portion 130 may be manufactured so that the tester 300 can be inserted into and detached from the device seating portion 130 or the tester 300 can be fixed integrally with the device seating portion 130.

The device seating portion 130 may include a device insertion groove 131, a fastening insertion groove 132, and a leg hole 133.

The device insertion groove 131 is a groove which a part of the tester 300 is inserted into or fixed integrally with, and is manufactured in the same shape as the outer shape of the tester 300.

Furthermore, the device insertion groove 131 may be manufactured to a depth such that the insertion hole 320 of the inserted or integrated tester 300 is exposed to the outside, and the rear surface of the inserted tester 300, that is, a surface where a lens part 310 (see FIG. 7) is formed, may be positioned up to the position of the mobile phone seating area 30 or higher by a predetermined length than the position of the mobile phone seating area 30. In the present disclosure, using a circular tester 300 as an example, the device insertion groove 131 is illustrated as a circular groove.

The fastening insertion groove 132 is formed so that the height adjustment portion 140 may be inserted and fixed into the device insertion groove 131. The fastening insertion groove 132 is a groove formed by protruding from a portion of the inner bottom surface of the device insertion groove 131 toward the rear side (lower side) of the body 110. A fastening rod 143 of the height adjustment portion 140 may be inserted into the fastening insertion groove 132. The leg hole 133 is a hole into which a plurality of rise-restricting legs 144 formed in the height adjustment portion 140 are inserted, and may be formed in the bottom surface of the device insertion groove 131.

The height adjustment portion 140 supports the tester 300 inserted into the device insertion groove 131. The height adjustment portion 140 may be lowered to the lower side of the body 110 by a force exerted by the mobile phone 400 when the mobile phone 400 is placed in the mobile phone seating area 30. If the mobile phone 400 is placed but no force is applied to press the mobile phone 400, the height adjustment portion 140 may not descend.

Meanwhile, a plurality of opposing fitting plates 161 or 162 may be formed in the rear surface of the body 110. The plurality of fitting plates 161 are provided to have the cup 40 fit therein. When a lower part of the cup 40 is fitted, a gap between the opposing fitting plates may be adjusted to match a diameter of the lower part. Similarly, when an upper part of the cup 40 is fitted, the gap may be widened to match the diameter of the upper part.

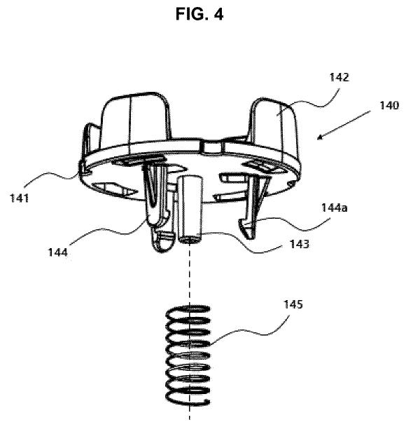

The height adjustment portion 140 will be described in more detail with reference to FIGS. 4 and 5. FIG. 4 is an exploded perspective view of a height adjustment portion in a tester stand according to an embodiment of the present disclosure, and FIG. 5 is a perspective view showing a height adjustment portion mounted on a tester stand according to an embodiment of the present disclosure.

A height adjustment portion 140 may include a support plate 141, a fixing plate 142, a fastening rod 143, a plurality of rise-restricting legs 144, and an elastic body 145.

The support plate 141 is manufactured in a plate shape and is positioned on a bottom surface of a device insertion groove 131. The support plate 141 is initially positioned apart from the bottom surface of the device insertion groove 131 when a tester 300 is not inserted. When the tester 300 is pressed by the mobile phone 400, the support plate 14 descends to the bottom surface and comes into close contact with the bottom surface. Afterwards, when a mobile phone 400 is removed from the stand 100, the support plate 141 returns to the initial position by the elastic body 145.

When the tester 300 is placed in the device seating portion 130, the tester 300 is positioned at the top. The support plate 141 may be circular overall but may also be manufactured in various shapes suitable for insertion into the device insertion groove 131.

The fixing plate 142 is formed by protruding vertically from an edge of the support plate 141 and contacts a side surface of the tester 300 positioned on the support plate 141 to fix the tester 300 in place. Meanwhile, if the device insertion groove 131 is manufactured to fix the inserted tester 300, the fixing plate 142 may be omitted.

The fastening rod 143 protrudes downward from the lower surface (rear surface) of the support plate 141, has a shape corresponding to the shape of the fastening insertion groove 132, and is inserted into the fastening insertion groove 132. For example, the fastening rod 143 may be in the shape of a rod or cylinder.

A plurality of rise-restricting legs 144 protrude downward from the lower surface of the support plate 141 and are positioned by passing through the leg holes 133 of the device seating portion 130. A catch 144a is formed at the end of each rise-restricting leg 144. The catch 144a engages with the leg hole 133 when the support plate 141 is raised by the elastic body 145, preventing the support plate 141 from rising further.

The elastic body 145 is fitted onto the fastening rod 143 and inserted into the fastening insertion groove 132. When a pressing force from the mobile phone 400 is applied, the elastic body 145 contracts to generate elasticity. When the pressing force of the mobile phone 400 is removed, the elastic body 145 raises the support plate 141 and the tester 300 with the elasticity. The elastic body 145 may be a spring, and may be manufactured in various shapes other than a spring.

FIG. 6 is a perspective view showing a state in which a cup is mounted on a rear surface of a tester stand according to an embodiment of the present disclosure. Referring to FIG. 6, a cup 40 is shown fitted into fitting plates 161 and 162 formed in a rear surface of a tester stand 100 according to an embodiment of the present disclosure. Here, the cup 40 is a container for holding a liquid substance sample, and the number ‘1’ may be marked on a lid of the cup 40.

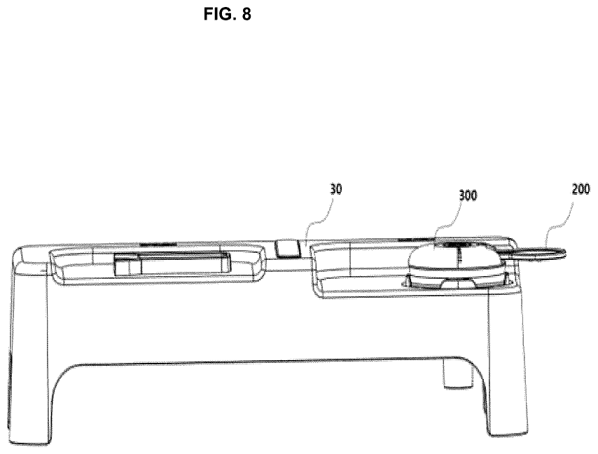

FIG. 7 is a perspective view showing a chamber and a tester mounted in a chamber area and a tester area of a tester stand according to an embodiment of the present disclosure, and FIG. 8 is a perspective view showing a state in which a chamber is inserted into a liquid substance tester in a tester stand according to an embodiment of the present disclosure.

Referring to FIGS. 7 and 8, a user inserts and places a chamber 200 into the device seating portion 130 to examine or observe a liquid substance sample. At this point, the protrusion 210 formed in the rear surface of the chamber 200 is fitted into the fitting groove 123.

Then, the user places the tester 300 in the device seating portion 130. At this point, the user positions the insertion hole 320 of the tester 300 in an open portion of the device task area 20 and places the tester 300 so that the lens part 310 faces upward.

After placing the chamber 200 and the tester 300 on the stand 100, the user performs a task of collecting a liquid substance sample from the cup 40, containing the liquid substance sample, based on the numbers indicating the task sequence, injecting the liquid substance sample into the chamber 200 placed in the chamber seating portion 120 of the stand 100, and inserting the chamber 200 containing the liquid substance sample into the tester 300 placed in the device seating portion 130 of the tester 100.

Here, the tester 300 placed in the device seating portion 130 is positioned in proximity to the mobile phone seating area 30. In this case, being positioned in proximity to the mobile phone seating area 30 may indicate that the tester 300 is at the same height as the mobile phone seating area 30, lower than the mobile phone seating area 30 by a predetermined value, or higher than the mobile phone seating area 30 by a predetermined value.

FIG. 9 is a perspective view showing a state in which a mobile phone with a flat lens is placed on a tester stand according to an embodiment of the present disclosure. Referring to FIG. 9, when a mobile phone 400 with a non-protruding lens is placed on the mobile phone seating area 30 of a tester stand 100 with a chamber 200 and tester 300 mounted on the test stand 100 according to an embodiment of the present disclosure, the mobile phone 400 is positioned in proximity to or in contact with the tester 300. When the user adjusts the position of the mobile phone 400, the lens of the mobile phone 400 is positioned over the lens part 310 of the tester 300. Afterwards, the user is able to observe a liquid substance sample contained in the tester 300 through a screen of the mobile phone 400.

At this point, if the rear surface of the tester 300 is positioned slightly higher than the mobile phone seating area 30, the tester 300 may be slightly lowered by the mobile phone 400. However, if the rear surface of the tester 300 is positioned at the same level as or slightly lower than the position of the mobile phone seating area 30, the tester 300 does not move.

FIG. 10 is a perspective view showing a state in which a mobile phone with a protruding lens is placed on a tester stand according to an embodiment of the present disclosure. Referring to FIG. 10, when a mobile phone 400 having a protruding lens 410 is placed on a mobile phone seating area 30 while a chamber 200 and a tester 300 are placed on a tester stand 100 according to an embodiment of the present disclosure, the lens 410 of the mobile phone 400 presses the tester 300, causing the tester 300 to be lowered. And when the user adjusts the position of the mobile phone 400, a lens of the mobile phone 400 aligns with a lens part 310 of the tester 300.

Afterwards, the user is able to observe a liquid substance sample contained in the tester 300 through a screen of the mobile phone 400.

According to an embodiment of the present disclosure, it is possible for a user to easily place a liquid substance sample in a chamber and easily insert the chamber into a tester.

In addition, it is possible for the user to observe the liquid substance sample inside the tester through a lens of a mobile phone, without holding the mobile phone or the tester.

The technical features disclosed in each embodiment of the present disclosure are not limited to a corresponding embodiment, and unless incompatible with each other, the technical features disclosed in each embodiment may be applied in combination to other embodiments.

Therefore, although each embodiment is described mainly about an individual technical feature, the technical features of the embodiments of the present disclosure may be applied in combination, unless incompatible with each other.

The present disclosure is not limited to the above-described embodiments and the accompanying drawings, and various modifications and changes may be made in view of a person skilled in the art to which the present disclosure pertains. Therefore, the scope of the present disclosure should be determined by the scope of the appended claims, and equivalents thereof.

What is claimed is:

1. A tester stand comprising:
a body having a predetermined height, and a chamber task area and a device task area each formed in a groove shape engraved in an upper surface of the body;
a chamber seating portion formed by protruding from a bottom surface of the chamber task area and in which the chamber is placed;
a device seating portion having a device insertion groove formed in a bottom surface of the device task area so that a part of the tester is seated therein, and having a downward fastening insertion groove on the bottom surface of the device insertion groove; and
a height adjustment portion fitted into the fastening insertion groove so that a support plate is positioned on the bottom surface of the device insertion groove, wherein when an external force is applied, the support plate descends to press against the bottom surface, and when the external force is removed, the support plate rises to return to an initial position thereof.

2. The tester stand of claim 1, wherein a non-slip pad portion is formed in the upper surface of the body to prevent the mobile phone from slipping.

3. The tester stand of claim 1, wherein the height adjustment portion comprises:
a support plate positioned on the bottom surface of the device insertion groove and in close contact with the inserted tester to support the tester;
a fastening rod protruding downward from a lower surface of the support plate and inserted into the fastening insertion groove;
a plurality of rise-restricting legs protruding downward from the lower surface of the support plate, penetrating through the bottom surface of the device seating portion, and having catches formed at ends of the plurality of rise-restricting legs to engage with a bottom of the device seating portion when the support plate is raised; and
an elastic body fitted onto the fastening rod and inserted into the fastening insertion groove 132, wherein the elastic body contracts to generate elasticity when external force is applied, whereas the elastic body raises the support plate with the elasticity when the external force is removed.

4. The tester stand of claim 3, wherein the height adjustment portion further comprises a fixing plate that protrudes vertically from an edge of the support plate and contacts a side surface of the tester positioned on the support plate to fix the tester.

5. The tester stand of claim 1, wherein the chamber seating portion comprises:
a chamber seating area formed by protruding from the bottom surface of the chamber task area and having a shape identical to an outer shape of the chamber; and
a fixing frame formed by protruding along an edge of the chamber seating area to a predetermined height.

6. The tester stand of claim 5, wherein the chamber seating portion further comprises a fitting groove formed in a shape of a groove in the chamber seating area and into which a protrusion formed in the rear surface of the chamber is fitted.

7. The tester stand of claim 5, wherein the chamber seating area is coated with a first color different from a color of the chamber task area, or a tape or film having the first color is attached to the chamber seating area.
